(19) United States
(12) Patent Application Publication
(10) Pub. No.: US 2004/0076652 A1
(43) Pub. Date: Apr. 22, 2004

(54) Vesicle forming skin oils comprising w/o-emusifiers with an hydrophilic-lipophilic balance of 2-6, method for the production and the use thereof

(76) Inventors: Valentina Paspaleeva-Kuhn, Frankfurt (DE); Simone Schatschneider, Wiesbaden (DE); Rolf Beutler, Hochst/Hummetroth (DE)

Correspondence Address: WILLIAM COLLARD, COLLARD & ROE, P.C., 1077 NORTHERN BOULEVARD, ROSLYN, NY, 11576, US

(21) Appl. No.: 10/450,543

(22) PCT Filed: Nov. 2, 2001
(86) PCT No.: PCT/EP01/12709

(30) Foreign Application Priority Data
Dec. 15, 2000 (DE) 10062610.6

(51) Int. Cl. A61K 7/06; A61K 7/11

(52) U.S. Cl. 424/401; 424/70.13

(57) ABSTRACT

The invention relates to skin oils containing fat, containing one or more oil soluble components, one or more W/O emulsifiers with a hydrophilic-lipophilic balance of 2-6, preferably 5.9, one or more vesicle forming lipids and, optionally, one or more additives selected from etherical oils, antioxidants, perfumed materials, preservatives, active ingredients, UV filters, vitamins, consistency modulators and solubilizers. The invention also relates to the production and the use of said skin oil, particularly as a skin care oil, sport oil, massage oil or sun protection oil. Said skin oils can be applied on dry skin and particularly on wet skin. Said oil is self-emulsifying and spontaneously forms liposomes. Said oil also has the advantage as it can easily penetrate the skin without leaving a disturbing layer of fat thereon.

The present invention relates to fat-containing skin oils comprising one or more oil-soluble components, one or more W/O emulsifiers with an HLB value of 2-6, preferably 2-5.9 and one or more vesicle-forming lipids, and optionally one or more additives chosen from essential oils, antioxidants, perfume substances, preservatives, active ingredients, UV filters, vitamins, consistency modulators, solubilizers, to their preparation and also to their use as skin oil, in particular as skincare oil, skin protection oil, sport oil, massage oil, or sunscreen oil. These skin oils can be applied to dry skin and, in particular, to wet skin, where they have a self-emulsifying effect and spontaneously form liposomes. They thus have the advantages associated therewith (active substance carrier, construction of skin barrier) also as a result of the ready absorption into the skin without leaving behind an undesired greasy film on the skin.

Skin oils have established a firm place in the broad pallet of cosmetic care products. They have the task of compensating for the deficit of skin lipids, of making the skin soft and supple, of promoting skin elasticity or of protecting the skin through the formation of a hydrophobic film. For massages, they are used as lubricants.

By incorporating specific active ingredients, so-called skin function oils, e.g. sunscreen oils, sport oils, can be formulated.

These are liquid, oily preparations which are prepared by mixing various fats and oils, waxes, fatty acid esters and liquid hydrocarbons or silicone oils.

In addition, stabilizers, such as antioxidants, preservatives or specific active ingredients, such as UV filters, vitamins, essential oils or plant extracts, can be incorporated. The so-called hydrophilic oils additionally comprise an O/W emulsifier and can be washed off with water as a result of the formation of an O/W emulsion.

Such customary skin oils are also described in the monograph by K. Schrader, Grundlage und Rezepturen der Kosmetika [fundamentals and formulations of cosmetics], 2nd Edition (1989, Hüthig Buchverlag), pp. 525-528.

The “Handbuch der Kosmetika und Riechstoffe” [handbook of cosmetics and fragrances], Vol. III, 1973, pp. 482-489 (Dr. A. Hüthig Verlag Heidelberg) describes various skin oils. Examples of components which can be used are: non-drying oils, mineral oils of varying viscosity, isopropyl myristate and isopropyl palmitate, deodorized isopropyl oleate, isopropyl isostearate, liquid hexadecyl alcohol, hexadecyl isostearate, high-purity oleyl alcohol (HD-Eutanol), cetiol LG, cetiol HE, myritol 318, cremogen HP, Miglyol 812, cetiol B, oleyl oleate, decyl oleate (cetiol V), perhydrosqualene (Cosbiol), liquid acetoglycerides, amerchol L-101, Acetulan (American cholesterol), lanolin derivatives (e.g. liquid lanolin; Modulan etc.), isohexadecyl palmitostearate (Wickenol 1109-H), cetyl polypropylene glycol ether (P.R.O. cetyl alcohol 30), lanolin alcohol polypropylene glycol ether (P.R.O. lanolin alcohol 30), hexadecyl polypropylene glycol ether (P.R.O. hexadecyl alcohol 30), silicone oils, propylene glycol diperlargonate (Emery 3771-D).

The additives described are, especially generally, the following “active ingredients”:

lecithins, liquid lanolin and lanolin derivatives, oily drug extracts (camomile, arnica, hypericum, calendula), oily skin extracts (and gland extracts), camphor, menthol, camphor phenoxide, ichthyol (oil-soluble), aluminum stearate (also as thickener), liquid polyethylene glycol mono fatty acid esters, vitamins (A, D₃, E etc.), β-carotene, chlorophyll, oil-soluble.

In particular, a face oil consisting of olive oil, eutanol G, neoba O, lecithin oil, purcellin oil, perfume, color, preservatives is disclosed. Also described are various massage oils, body oils and sport oils which comprise predominantly paraffin oil (cf. pp. 487-488).

Also described are skin oils which can be washed off which have the abovementioned O/W emulsifiers.

Schrader, K. H., Grundlagen und Rezepturen der Kosmetika [Fundamentals and Formulations of Cosmetics], 1989, pp. 525-528, describes skin oils which can have, as oil component, Cetiol V, and active ingredients and lecithin.

DE 31 41 761 describes the use of glucose derivatives in, for example, oils, where the glucose derivatives are used as surface-active substances.

SÖFW, 115 (1989), pp. 344-350 discloses general bases for the preparation of anhydrous products and also describes skin oils. These are characterized as lipid-containing anhydrous liquid preparations [lacuna] can additionally have an O/W emulsifier with an HLB value greater than 8. The oil components described here are customary paraffins or e.g. octyldodecanol, myritol 318, cetiol, Miglyol and also optionally liquid lanolin, phenylmethylsilicone. Reportedly, these skin oils are, in particular, water-repellent (cf. p. 345, point 2.1.1.2.6).

SÖFW, 98 (1972), pp. 889-891 describes hydrophilic oils which, as well as the customary oil components, have nonionic emulsifiers, such as polyoxyethylated laurates/oleates and corresponding sorbitan derivatives with an HLB value of 8-11. Such polyoxyethylated products are, as is known, O/W emulsifiers and reportedly form O/W emulsions. These also include combinations of emulsifiers with a total HLB value of from 8 to 11, such as, for example, the combination of Arlacone® T (HLB=9.2), Tween® 85 (HLB=11.0) and Span® 85 (HLB=1.8).

EP-A 0 467 218 describes lipid combinations which have at least two of the following components: unsaturated fatty acids and/or their tocopheryl esters; n-alkanes; squalene; cholesterol and/or wool wax alcohol; triglyceride; wax esters. Particular preference is given to combinations of unsaturated fatty acids/cholesterol/wool wax alcohol. These combinations can reportedly be used as they are or in a hydrous base spread on the skin.

Liposome-containing care and cleansing formulations are described in EP No. 0 523 418, EP 0 557 825, DE 198 54 827. These are generally hydrogels, O/W emulsions or washing products. For example, EP-B 0 557 825 describes oil-containing bath and shower additives consisting of oil components, vesicle-forming lipids and O/W surfactants with an HLB value of 6-13. These compositions are cleansing products. EP-B 0 523 418 relates to liposome-containing creams with oil phase and water phase and certain polyoxyethylated surfactants as emulsifiers with HLB values of 9-13. However, these do not achieve the care action of a skin oil. Furthermore, in the case of skincare products, vesicles are usually only prepared by complex processes and incorporated into the desired preparation. Stability problems may arise, particularly in the case of emulsions.

DE 198 54 827 relates to surfactant-containing cleansers which are oil-free and have sterols as liposome formers.

An object of the present invention is therefore to provide oil-containing products which can also be applied to wet skin, have a self-emulsifying action and simultaneously form liposomes, so that a care effect as during rubbing in is also achieved without the skin being subjected beforehand to drying and without the greasy film customary with skin oils being present, which is or can only be removed after a prolonged time or, where appropriate, only by rubbing or washing off.

This object is achieved according to the invention by skin oil compositions comprising one or more oil-soluble components and one or more W/O emulsifiers with an HLB value of 2-6, in particular 2-5.9, and one or more vesicle-forming lipids, and optionally one or more additives chosen from active ingredients, antioxidants, perfume substances, preservatives, UV filters, vitamins, dyes, solubilizers, consistency modulators, with the exception of compositions comprising cholesterol and/or wool wax alcohol.

If said oil components are combined with the described W/O emulsifiers and the vesicle formers, it has been found that, upon application, in particular to wet skin, the oil has a self-emulsifying effect and thus is easy to apply and also is without an undesired greasy film. This is surprising since here no apparatuses customary for the preparation of emulsions, in particular stirrers or high-speed homogenizers, are used. In addition, liposomes surprisingly form. This could not be expected since here only a small amount of water is present in total (for example 1:1 compared to the oil), whereas for analogous applications in the shower or during bathing, far higher amounts of water (for example 10 times, or 100 times to 1 000 times, respectively, the amount), based on the oilbath, are present, and vesicles normally require such a much higher amount of water in order to form.

In the skin oil composition according to the invention, the oil-soluble component(s) is/are present in an amount of 50-99%, the W/O emulsifier(s) is/are present in an amount of 0.5-10%, the vesicle-forming lipid(s) is/are present in an amount of 0.01-10% and the additive(s) are present in an amount of 0-40%, in particular 0.1-40%, preferably 0.5-20% and very particularly preferably 1-15% in total.

According to the invention, the emulsifier(s) can be present in amounts of 0.5-10%, in particular from 1.0-8% and very preferably 2-5% and the vesicle formers can be present in an amount of in particular 0.01-10%, preferably 0.5-5% and in particular 1-3%. The oil component(s) is/are present here in amounts of 50-99%, in particular 70-97% and especially of 85-97%. In a particularly preferred embodiment, the compositions can one or more W/O emulsifiers (alone or in a mixture with one another) with an HLB value of in particular 3-5.5 and very particularly from 3.5-5.5 be used [sic].

The amounts correspond to those given above.

In a further preferred embodiment, the oil component(s) is/are present in an amount of 85-97%, the emulsifier(s) is/are present in an amount of 1-10%, in particular 2-6% and very particularly 1-5%, the vesicle formers are present in an amount of 0.5-5% and the additives are present in an amount of 1-20%, in particular 1-15%.

Particularly preferred emulsifiers are the following, the HLB value being given in brackets in each case:

### Sorbitan Derivatives

Sorbitan esters, such as sorbitan oleate (Span® 80, HLB=4.5), sorbitan stearate (HLB =5.0), sorbitan sesquioleate (Crill® 43, HLB=3.7), sorbitan isostearate (Crill® 6, HLB=4.7), sorbitan tristearate (Crill® 35, HLB=2.1);

### Polyethoxylated Products

Polyethoxylated fatty acids and fatty alcohols, such as PEG-2 oleate (HLB=5.0), PEG-4 distearate (HLB=3.0), PEG-2 stearate (HLB=4.4), ceteareth-3 (Volpo® CS3, HLB=5.0), ceteth-2 (Volpo® C2, HLB=5.3);

Ethoxylated triglycerides, such as PEG-5 castor oil (HLB=3.9), PEG-6 diricinoleate (HLB=5.0), PEG-7 hydrogenated castor oil (Cremophor® WO 7, HLB=5.0);

### (Poly)glyceryl Derivatives

Polyglyceryl esters, such as polyglyceryl-3 diisostearate (Lameform® TGI, HLB=3.5), polyglyceryl-2 dipolyhydroxystearate (Dehymuls® PGPH, HLB=3.5), diisostearoyl polyglyceryl-3 diisostearate (Isolan® PDI, HLB ca. 5), polyglyceryl-3 oleate (Isolan® Go 33, HLB ca. 5), polyglyceryl-3 dioleate (Cremophor® GO 32, HLB ca. 5), polyglyceryl-4 isostearate (Isolan® GI 34, HLB ca. 5);

glycerin esters, such as glyceryl ricinoleate (Cithrol® GMR N/E, HLB=2.7), glyceryl laurate (Cithrol® GML N/E, HLB=4.9), glyceryl dioleate S/E (° Cithrol® GDO S/E, HLB=2.9);

### Polyol Esters

Polyol esters, such as glycol oleate S/E (Cithrol® EGMO S/E, HLB=2.7), glycol ricinoleate (Cithrol® EGMR S/E, HLB=2.0), glycol dilaurate S/E (Cithrol® EGDL S/E, HLB=2.0), propylene glycol ricinoleate (Cithrol® PGMR S/E, HLB=3.6), propylene glycol laurate (Cithrol® PGML N/E, HLB=2.7);

### Glucose Derivatives

Glucose esters, such as methyl glucose dioleate (Isolan® DO, HLB ca. 5), methyl glucose isostearate (Isolan® IS, HLB ca. 5);

### Pentaerythritol Derivatives

Pentaerythritol fatty acid esters, e.g. pentaerythritol monolaurate (HLB=4.8), pentaerythritol monotallate (HLB=4.0) or mixed esters, e.g. with citric acid fatty alcohol esters such as Dehymuls® E (dicocoyl pentaerythrityl distearyl citrate, sorbitan sesquioleate, Cera alba, aluminum stearate, HLB=4.0), Dehymuls® F (dicocoyl pentaerythrityl distearyl citrate, Cera microcristallina, glyceryl oleate, aluminum stearate, propylene glycol (HLB=4.0);

### Alkylphenols

Alkyphenols, e.g. nonoxynol-2 (HLB ca. 4.5);

### Polymers

Polymers such as polyoxypropylene-polyoxyethylene block polymers (INCI name: Poloxamers), e.g. Pluronic® PE 3100 (HLB=4.5), Pluronic® PE 6100 (HLB=3.0) or PEG-30 dipolyhydroxystearate (Arlacel® P 135, HLB ca. 5.5);

### Siloxane Derivatives

Polysiloxane copolymers, in particular those mentioned below from this group.

Preference is given to ethoxylated products, such as fatty acids and triglycerides, such as PEG-2 oleate, PEG-7 hydrogenated castor oil (Cremophor® WO 7), (poly)glyceryl ester and glucose ester, and polysiloxane copolymers or mixtures thereof.

Particular preference is given to polysiloxane copolymers, such as polysiloxane-polyether copolymers, in particular polysiloxane-polyalkyl-polyether copolymers, such as cetyl dimethicone copolyol (Abil® EM 90., HLB ca. 5), laurylmethicone copolyol (Dow Corning® Q2-5200, HLB ca. 4).

Also particularly preferred are emulsifier mixtures containing siloxane derivatives, such as standard commercial Abil® WE 90 (HLB ca. 5) consisting of cetyl dimethicone copolyol, polyglyceryl-4 isostearate and hexyl laurate.

Alternatively, it is also possible to combine individual emulsifiers from in each case one of the said groups or else from different groups, where combinations of polysiloxane copolymers and polyglyceryl esters are preferred.

Suitable as oil components are customary, preferably liquid, lipids, these may be present individually or in a mixture. In particular, the following groups and examples thereof are suitable:

### Hydrocarbons

Hydrocarbons such as squalene, squalane, in particular also liquid paraffins, isoparaffins, dioctylcyclohexane (Cetiol® S), isohexadecane (Arlamol® HD);

Fatty alcohols, such as oleyl alcohol, octyldodecanol (Eutanol® G);

Fatty acid esters, e.g. isopropyl fatty acid esters (palmitate, myristate, isostearate, oleate), decyl oleate (Cetiol® V), hexyl laurate, C12-15 alkylbenzoate (Finsolv® TN), dicaprylyl carbonate (Cetiol® CC), diesters, such as dibutyl adipate (Cetiol® B), propylene glycol dipelargonate, branched fatty acid esters, such as PCL-liquid® (cetearyl octanoate) or mixtures such as Cetiol® PGL (hexyldecanol and hexyldecyl laurate).

Fatty alcohol ethers, such as dicaprylyl ether (Cetiol® OE).

Polyol fatty acid esters, such as Cetiol® HE (PEG-7 glyceryl cocoate).

Triglycerides, in particular medium-chain (neutral oils), such as caprylic/capric triglyceride (Miglyol® 810, 812), and their polyol esters, such as propylene glycol dicaprylate/dicaprate (Miglyol® 840).

Natural fats and oils, such as sunflower oil, soybean oil, peach kernel oil, apricot kernel oil, grape seed oil, castor oil, groundnut oil, almond oil, clove oil, wheat germ oil, avocado oil.

Natural liquid waxes, e.g. jojoba oil or its substitute oleyl erucate (Cetiol® J 600).

Silicone oils and silicone waxes, e.g. polydimethylsiloxanes, such as Dow Corning Fluid® 200 (dimethicone), cyclomethylsiloxanes, such as Dow Corning Fluid® 345 (cyclomethicone), phenylmethylpoly-siloxanes, such as phenyl dimethicone (Abil® AV 8853) or alkyl-polymethylsiloxane copolymers, such as cetyl dimethicone (Abil® Wax 9801), stearyl dimethicone (Abil® Wax 9800), dialkoxydimethylpolysiloxanes, such as stearoxy dimethicone (Abil® Wax 2434), behenoxy dimethicone (Abil® Wax 2440).

Particularly preferred oil components are liquid paraffins, fatty acid esters, such as isopropyl palmitate or myristate, medium-chain triglycerides, such as the abovementioned Miglyols and said natural fats and oils, in particular sunflower oil, soybean oil, peach kernel oil, apricot kernel oil, in particular mixtures thereof, and also jojoba oil, its mixtures with the abovementioned components, where, in each case, ca. 1-40% of individual component, based on the total amount, are present, or fatty alcohol ethers or fatty alcohols or mixtures thereof or hydrogenated oils such as perhydrosqualene, individually or combined.

Particularly preferably, said liquid paraffins and said triglycerides are also in combination with one another. Also suitable are the abovementioned dioctylcyclo-hexanes, isohexadecanes, in particular the latter, silicone oils and silicone waxes, especially also combinations thereof and also with the abovementioned paraffins and triglycerides. These can then in particular also be combined with the natural fats, oils and waxes.

Suitable vesicle-forming lipids are, in particular, substances known for this purpose (cf.. DE 42 05 548 C2), in particular phospholipids such as lecithin (egg or soybean lecithin), e.g Phosal® 50 SA (about 50% soybean lecithin), phosphatidyl choline, serine or diethanolamine and mixtures thereof.

Further suitable components for the preparation of vesicles are sphingolipids (e.g. ceramides, cerebro-sides, sphingosine, sphingomyelin), phytosterols (essentially mixtures of β-sitosterol, campesterol and stigmasterol) and derivatives thereof, in particular ethoxylates such as Generol® 122 E 5 (PEG-5 Soybean Sterol), Gererol® R E5 (PEG-5 Rapeseed Sterol).

Also suitable are polyethoxylated fatty alcohols and fatty acids having preferably 1-4 EO with an HLB value of from 2 to 6, where the lipophilic radical preferably consists of C₁₆ to C₁₈-alkyl chains, polyglycerol alkyl ethers, glucosyl dialkyl ethers, sucrose diesters, collagen hydrolysate esters, quaternary ammonium compounds and poloxamers.

Particularly preferred vesicle formers are phospholipids such as egg and/or soybean lecithin, (Phosal® 50 SA), phosphatidyl choline, ceramides, phytosterols and ethoxylated derivatives thereof with a degree of ethoxylation of from 5 to 16 and polyoxyethylated fatty alcohols with an HLB value of 2-6 or combinations thereof, in particular of phytosterols of the abovementioned type with ethoxylation products thereof.

According to the invention, it is preferred if the oil composition, as well as having the main components oil/emulsifier/vesicle former, has as additional substances active ingredients, which also include essential oils and plant extracts, preservatives and vitamins.

Suitable vitamins are, in particular, vitamin A, E, C and derivatives thereof, e.g. retinol, retinol acetate, or retinol palmitate, carotinoids, tocopherol or tocopherol acetate, ascorbyl palmitate.

The active ingredients are preferably chosen from essential oils and terpenes, e.g. rosemary oil, orange oil, lavender oil, lime oil, cinnamon oil, geranium oil, cedarwood oil, rosewood oil, valerian oil, ylang ylang oil, eucalyptus oil, mint oil, lemongrass oil, cypress oil, niaouli oil, spruce needle oil, pine needle oil, camphor, menthol.

In addition, it is also possible here to use essential unsaturated fatty acids and esters thereof, e.g. linoleic acid or linolenic acid, glyceryl linoleate, glyceryl linolenate.

Instead of or as well as the abovementioned active ingredients, it is also possible to use circulation-promoting substances, e.g. nicotinic acid derivatives, such as methyl or tocopheryl nicotinate, alpha- and betahydroxy acids and derivatives thereof, e.g. salicylic acid, isopropylbenzyl salicylates, C12-13 alkyl lactates (Cosmacol® ELI) or else antiphlogistic and antibacterial substances, such as glycyrrhizinic acid or glycyrrhetinic acid and derivatives thereof, e.g. stearyl glycyrrhetinate, panthothenic acid derivatives, e.g. panthenyl triacetate, allantoin, bisabolol, azulenes, e.g. chamazulenes or guaiazulene, triclosan, chlorhexidine derivatives and/or antidandruff agents, e.g. climbazole or piroctone olamine.

In addition, repellents, such as N,N-diethyl-m-toluamide or dimethyl phthalate; or substances with an antioxidative and cell-protecting effect, such as flavonoids, e.g. rutin, ferulic acid and esters thereof or coenzyme Q 10, can also be used as effective additives in the skin oils according to the invention.

It is also possible to use plant extracts, which themselves may also be active ingredients, such as, for example, Aloe vera extract, lime blossom extract, Centalla asiatica extract, ivy leaf extract, can be used as effective additives [sic].

Said components can also be combined, depending on the desired intended use. For example, combinations of aromatherapeutically effective essential oils, such as rosemary oil, lime oil and/or lemongrass oil, or valerian oil, lavender oil and/or ylang ylang oil or circulation-promoting substances, such as rosemary oil and methyl nicotinate, in particular, are preferred.

Particularly preferred active ingredients are chosen from essential oils and terpenes, plant extracts, circulation-promoting substances, antiphlogistic and antibacterial substances, vitamins, essential unsaturated fatty acids or mixtures thereof.

Further particularly suitable additives from the group of active ingredients, which may also be essential oils as described above and can also represent perfume substances at the same time, are, in particular, mint oil, lime oil, orange oil, juniper oil, valerian oil, eucalyptus oil, thyme oil, palmarosa oil, rosemary oil, lavender oil, menthol, ginger extract, lime blossom extract, marigold extract, algae extract, Aloe vera extract, Echinacea extract, ivy leaf extract, hydroxyethyl salicylate, methyl salicylate, nicotinic esters or combinations thereof, such as, for example, orange oil, lavender oil, palmarosa oil and valerian oil or juniper oil, hydroxyethyl salicylate and methyl nicotinate. In this connection, depending on the intended effect, such as, for example, improvement in the hair structure, increase in circulation, tension relief, aromatherapy or the like, a suitable combination of active ingredients can be added. Where appropriate, further refatting agents, such as fatty acid glycerides and ethoxylates thereof, e.g. PEG-6 caprylic/capric glycerides (Softigen® 767), can be added.

It is, moreover, preferred if the skin oil composition comprise [sic], additionally or alternatively to the three abovementioned addition variants, as additive, those chosen from antioxidants, perfume substances, dyes and/or UV filters.

The antioxidants can then preferably be chosen from butylhydroxytoluene, butylhydroxyanisole, ascorbyl palmitate, tocopherol, possibly in combination with synergistic agents, such as in Controx® VP (tocopherol, lecithin, ascorbyl plamitate, hydrogenated palm glycerides citrate), gallic alkyl esters, such as octyl, dodecyl and cetyl gallate or combinations thereof.

Particularly preferred perfume substances are also standard commercial perfume compositions as well as the essential oils specified above under “active ingredients”.

Preferred dyes are, for example, Patent Blue, Amidoblue, Orange RGL, cochenille red, quinoline yellow, in particular to connect with a suitable solubilizer. This can be chosen, in particular, from ethanol and isopropanol, e.g. in amounts of 5-30%, in particular 5-15%.

Particularly preferred dyes are carotinoids, e.g. alpha- or beta-carotene or azulenes, such as chamazulene or guaiazulene.

Suitable UV filters are are [sic] oil-soluble UVB, UVA and broadband filters of the following type:

UV-B filters: cinnamic esters, e.g. octyl methoxycinnamate (Eusolex® 2292, Neo Heliopan® AV, Parsol® MCX), isoamyl p-methoxycinnamate (Neo Heliopan® Galanga) and 4-methylbenzylidenecamphor (Eusolex® 6300), paraaminobenzoic acid and esters, such as 2-ethylhexyl N,N-dimethyl-4-aminobenzoate (Eusolex® 6007, octyl dimethyl PABA), homomenthyl salicylate (homosalate, Eusolex® HMS), octyl salicylate (Neo Heliopan® OS), octocrylene (Neo Heliopan® 303), butylmethoxydibenzoylmethane (Eusolex® 9020); UVA and UVB filters for broadband absorption, such as benzophenone-3 (Neo® Heliopan BB, Eusolex® 4360); UV-A filters, such as methyl anthranilate (Neo Heliopan® MA).

Particular preference is given to octyl or isoamyl p-methoxycinnamate, octocrylene, 4-methylbenzy-lidenecamphor, homosalate and/or methyl anthranilate and/or benzophenone-3.

Suitable preservatives are iodopropynyl butylcarbamate, phenoxyethanol and further customary preservatives, such as, for example, sorbic acid and dehydracetic acid and salts thereof, methyldibromoglutanonitrile [sic], etc. or combinations thereof, or other acids, such as benzoic acid or salicylic acid, or benzyl alcohol or esters, such as p-hydroxybenzoic esters, e.g. methyl-, ethyl-, propyl-, butyl-, isobutylparaben, preferably methyl- or propylparaben or mixtures thereof or climbazole or suitable combinations of said substances, such as, for example, methylparaben, propylparaben and sorbic acid.

The emulsifier-containing skin oil compositions according to the invention have a particular primarily self-emulsifying effect, in particular when applied to wet skin, coupled with better moisture storage, better rubbing in to the skin without an unpleasant greasy film feel, which results in particular from the combination according to the invention of oil component and W/O emulsifier.

Preferred combinations of oil/emulsifier comprise:

paraffin oil (20-40%) and/or Miglyol 812 (20-30%) and/or isopropyl palmitate (15-25%); or else additionally to this jojoba oil (1-5%) and/or peach kernel oil (3-5%) and as emulsifier Abil® EM 90 and/or Abil® WE 09.

For this purpose it is possible to use the above-mentioned vesicle formers, in particular Phosal® 50SA, and additives, in particular said combinations, and very particular preference is given to using essential oils, perfume oils and/or circulation-promoting substances and also plant extracts and UV filters.

If the oil according to the invention have [sic] a relatively thin-liquid consistency, 5-30%, preferably 5-15%, of ethanol or isopropanol can also be added as consistency modulator, in particular ethanol. These can also serve as solubilizers for other substances.

Conversely, the consistency modulator added may also be thickeners and/or substances for improving the feel on the skin or the water resistance, such as, for example, polyethylene waxes with a molar mass of 1500 to 20,000, such as Lunacera® PA Paste, Aerosil®, modified montmorillonite, such as Miglyol® Gel B (caprylic/capric triglycerides, stearalkonium hectorite, propylene carbonate), aluminum soaps, and their modifications, e.g. aluminum/magnesium hydroxide stearate, alkylated polyvinylpyrrolidones, such as Antaron® V-216 (PVP/hexadecane copolymer) and Antaron® V-220 (PVP/eicosene copolymer).

If certain purposes are intended, e.g. skincare oil, sport oil or sunscreen oil or massage oil, the additives are chosen accordingly, e.g. for skincare oil, vitamins, plant oils and/or plant extracts, for sport oil, circulation-promoting substances, such as methyl nicotinate and/or hydroxyethyl salicylate and/or essential oils, e.g. rosemary oil for sunscreen oil, UV-A, UV-B and broadband filters, and vitamins, such as vitamin E, vitamin C for massage oil, essential oils which have an aromatherapeutic and/or circulation-promoting effect, such as orange oil, lavender oil, palmarosa oil, lime oil, lemongrass oil, and/or vitamin A, vitamin E where additionally preservatives, antioxidants and/or perfume substances may be present in the amounts stated.

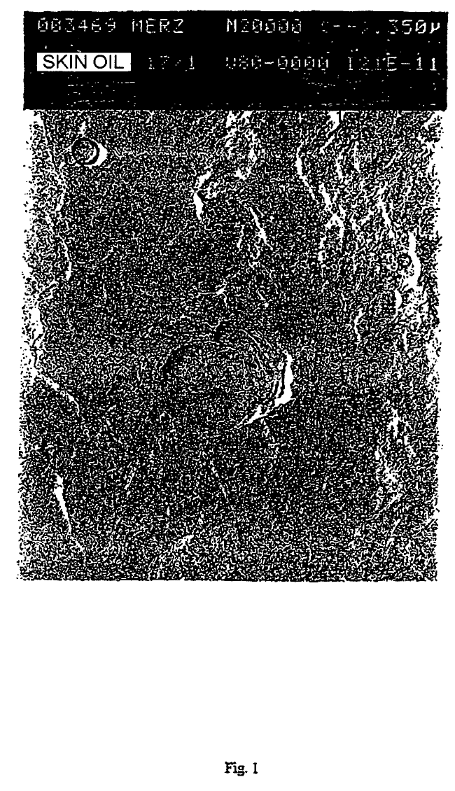

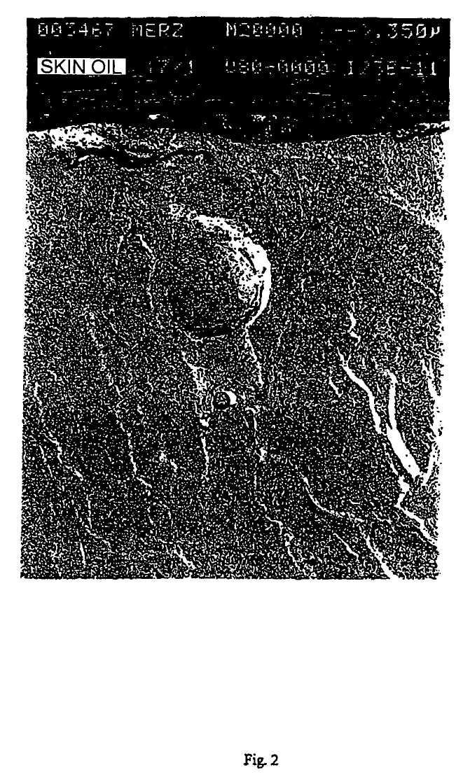

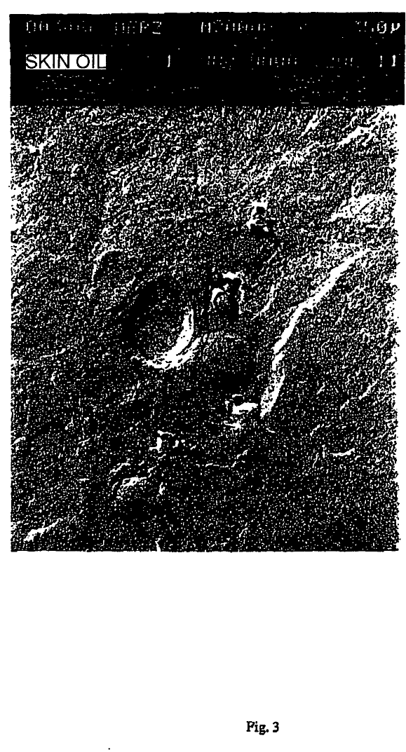

The invention is described in more detail by reference to Examples 1-5 below. In this connection, FIGS. 1-3 show the spontaneously formed liposomes following dilution with water in the ratio 1:1 at 33° C. upon application of the compositions as in Example 1 in a transmission electron microscope following freeze fracture.

The compositions according to the invention were prepared by stirring the liquid oils (if present) in a suitable vessel at room temperature until homogeneous, where necessary heating them to 40-90° C. in order to melt any solid waxes or fats present. The emulsifier(s) were then incorporated.

After cooling to room temperature, in particular 25° C., the additives, where present, were incorporated and stirred to homogeneity.

Where necessary, solid components can also be heated beforehand in a suitable manner, and then the nonsolid constituents be added at a suitable temperature (25-50° C., in particular 25-40° C.).

### EXAMPLE 1

### Skin Oil

| Raw materials | 100% formulation |
|---|---|
| Miglyol 812 | 36.80 |
| Peach kernel oil | 3.00 |
| Jojoba oil | 1.00 |
| Tocopherol acetate | 1.10 |
| Paraffin oil, thick-liquid | 28.40 |
| Isopropyl palmitate | 25.00 |
| Abil ® EM 90: cetyldimethicone copolyol | 2.00 |
| Phosal ® 50 SA: 50% lecithin | 2.00 |
| Perfume oil | 0.70 |
| TOTAL | 100.00 |

Notes:
Abil ® EM 90: polysiloxane-polyalkyl-polyether copolymer

### Example 2

### Skin Oil

| Raw materials | 100% formulation |
|---|---|
| Miglyol 812 | 36.12 |
| Peach kernel oil | 4.00 |
| Tocopherol acetate | 1.10 |
| Paraffin oil, thick-liquid | 28.40 |
| Isopropyl palmitate | 25.00 |
| Abil ® EM 90 | 2.00 |
| Phosal 50 SA: 50% lecithin | 2.00 |
| Valerian oil | 0.08 |
| Cedarwood oil | 0.20 |
| Rosewood oil | 0.10 |
| Perfume oil | 1.00 |
| TOTAL | 100.00 |

### EXAMPLE 3

### Oil-Based Skin Sunscreen Spray

| Raw materials | 100% formulation |
|---|---|
| Paraffin oil, thin-liquid | 50.50 |
| Finsolv TN: C12-C15-alkyl benzoate | 10.00 |
| Cetiol ® OE: dicaprylyl ether | 10.00 |
| Miglyol 812 | 20.00 |
| Abil ® EM 90 | 2.00 |
| Phosal 50 SA: 50% lecithin | 2.00 |
| UV filter: Neo Heliopan AV; octyl | 5.00 |
| methoxycinnamate | |
| Perfume oil | 0.50 |
| TOTAL | 100.00 |

### EXAMPLE 4

### Skin Oil

| Raw materials | 100% formulation |
|---|---|
| Miglyol 812 | 37.00 |
| Peach kernel oil | 3.00 |
| Jojoba oil | 1.00 |
| Tocopherol acetate | 1.10 |
| Paraffin oil, thick-liquid | 28.40 |
| Isopropyl palmitate | 25.00 |
| Dehymuls ® PGPH: polyglyceryl-2 | 2.00 |
| dipolyhydroxystearate | |
| Phosal ® 50 SA: 50% lecithin | 2.00 |
| Perfume oil | 0.50 |
| TOTAL | 100.00 |

### EXAMPLE 5

### Skin Oil

| Raw materials | 100% formulation |
|---|---|
| Miglyol 812 | 37.00 |
| Peach kernel oil | 3.00 |
| Tocopherol acetate | 1.10 |
| Paraffin oil, thick-liquid | 28.40 |
| Isopropyl palmitate | 26.00 |
| Isolan ® PDI: diisostearylpolyglyceryl-3 | 2.00 |
| diisostearate | |
| Phosal ® 50 SA: 50% lecithin | 2.00 |
| Perfume oil | 0.50 |
| TOTAL | 100.00 |

1) A vesicle-forming skin oil comprising one or more oil-soluble components, one or more W/O emulsifiers with an HLB value of 2-6 and one or more vesicle-forming lipids, and optionally one or more additives, chosen from active ingredients, antioxidants, perfume substances, preservatives, UV filters, vitamins, dyes, consistency modulators, solubilizers, with the exception of compositions comprising cholesterol and/or wool wax alcohol.

2) The skin oil as claimed in claim 1, characterized in that the oil-soluble component(s) is/are present in an amount of 50-99%, the emulsifier(s) is/are present in an amount of 0.5-10%, the vesicle-forming lipid(s) is/are present in an amount of 0.01-10% and the additive(s) is/are present in an amount of 0-40% in total.

3) The skin oil as claimed in claim 1 or 2, characterized in that the emulsifier(s) is/are chosen from sorbitan derivatives, polyethoxylated products, (poly)glyceryl derivatives, polyol esters, glucose derivatives, pentaerythritol derivatives, alkylphenols, polymers and siloxane derivatives or mixtures thereof.

4) The skin oil as claimed in any of claims 1-3, characterized in that the oil component(s) is/are chosen from hydrocarbons, fatty alcohols, fatty acid esters, fatty alcohol ethers, polyol fatty acid esters, triglycerides, natural fats, oils or natural liquid waxes or silicone oils or silicone waxes or mixtures thereof.

5) The skin oil as claimed in any of claims 1-4, characterized in that the oil component is chosen from liquid paraffin, isopropyl palmitate, medium-chain triglycerides, sunflower oil, soybean oil, peach kernel oil, apricot oil, jojoba oil, silicone oils or mixtures of the abovementioned components.

6) The skin oil as claimed in any of claims 1-5, characterized in that the W/O emulsifier(s) is/are present in an amount of 1.0-8%.

7) The skin oil as claimed in any of claims 1-6, characterized in that the W/O emulsifier(s) has/have an HLB value of 2-5.9.

8) The skin oil as claimed in claim 7, characterized in that the W/O-emulsifier(s) has/have an HLB value of 3.5-5.5.

9) The skin oil as claimed in any of claims 1-8, characterized in that cetyl dimethicone copolyol or cetyl dimethicone copolyol in a mixture with polyglyceryl-4 isostearate and hexyl laurate is present as emulsifier.

10) The skin oil as claimed in any of claims 1-9, characterized in that the vesicle-forming lipid is chosen [lacuna] phospholipids, such as egg and/or soybean lecithin, phosphatidyl choline, ceramides, phytosterols and ethoxylated derivatives thereof with a degree of ethoxylation of from 5 to 16 and polyoxyethylated fatty alcohols with an HLB value of 2-6 or combinations thereof, in particular of phytosterols of the type mentioned above with ethoxylation products thereof.

11) The skin oil as claimed in any of claims 1-10, characterized in that the skin oil has active ingredients, preservatives and vitamins as additives.

12) The skin oil as claimed in any of claims 1-11, characterized in that the skin oil has, as additives, those chosen from antioxidants, perfume substances, dyes, UV filters, as additives.

13) The skin oil as claimed in claim 11 or 12, characterized in that the active ingredients are chosen from essential oils and terpenes, plant extracts, circulation-promoting substances, antiphlogistics and antibacterial substances or mixtures thereof.

14) The skin oil as claimed in any of claims 1-13, characterized in that the perfume(s) is/are standard commercial perfume oil compositions, the preservative(s) is/are chosen from methylparaben or propylparaben or mixtures thereof and/or sorbic acid, the antioxidants are chosen from butylhydroxytoluene, butylhydroxyanisole, ascorbyl palmitate, tocopherol or mixtures thereof with synergistic agents, the vitamins are chosen from vitamin E, vitamin A, vitamin C and derivatives thereof.

15) The skin oil as claimed in any of claims 1-14, characterized in that the oil component(s) is/are present in an amount of 85-97%, the W/O emulsifier(s) is/are present in an amount to 2-6%, the vesicle-forming lipid(s) is/are present in an amount of 0.5-5% and the additives are present in an amount of 1-15%.

16) The skin oil as claimed in any of claims 1-15, characterized in that ethanol is present as solubilizer.

17) A process for the preparation of skin oils as claimed in any of claims 1-16, characterized in that the oil component(s) is/are mixed at room temperature and optionally heated to 40-90° C., the emulsifier(s) is/are incorporated, then the mixture is cooled to room temperature and then the additive(s) is/are incorporated.

18) The use of skin oils as claimed in any of claims 1-16 as skincare oil, skin protection oil, sport oil, massage oil or sunscreen oil.
